(19) United States
(12) Patent Application Publication
(10) Pub. No.: US 2024/0238113 A1
(43) Pub. Date: Jul. 18, 2024

(54) BUNION PATCH WITH NON-STICK & SINGLE-LAYERED CENTER

(71) Applicant: Daniel Jung, Issaquah, WA (US)

(72) Inventor: Daniel Jung, Issaquah, WA (US)

(21) Appl. No.: 18/097,156

(22) Filed: Jan. 13, 2023

(51) Int. Cl. A61F 5/30

(52) U.S. Cl. CPC A61F 5/30

(57) ABSTRACT

Apparatus and systems are disclosed for covering and protecting sensitive, sore, upset, and/or wounded areas of skin such that the pad or patch applied to these areas cannot and will not stick to the sensitive areas while at the same time covering these areas, or such that the pad or patch applied to these areas will be used to directly deliver any desired medication to these sensitive areas while covering and protecting them.

### CROSS-REFERENCE(S) TO RELATED APPLICATION(S)

None.

### TECHNICAL FIELD

This application relates generally to patches that cover specific parts of the human body. More specifically this application relates to pads, cushions, and patches designed to cover and protect foot bunions.

### BRIEF DESCRIPTION OF THE DRAWINGS

The drawings, when considered in connection with the following description, are presented for the purpose of facilitating an understanding of the subject matter sought to be protected.

FIG. 1 shows an exploded view of an example bunion patch, according to the disclosed apparatus and systems; and

FIG. 2 illustrates an example of the assemblage of the components of FIG. 1.

### DETAILED DESCRIPTION OF THE PREFERRED EMBODIMENT

While the present disclosure is described with reference to several illustrative embodiments described herein, it should be clear that the present disclosure should not be limited to such embodiments. Therefore, the description of the embodiments provided herein is illustrative of the present disclosure and should not limit the scope of the disclosure as claimed. In addition, while the following description references using limited number of fabrics, adhesives, and materials, it will be appreciated that the disclosure may include other woven and non-woven materials and adhesives to which the disclosed apparatus and methods also apply.

Foot care is crucial for overall well-being of a person. Foot pain or discomfort is often a sign of more serious health problems. Unfortunately, foot care products on the market fail to address the many multifaceted issues of their users. For example, the center section of the conventional bunion patches, which must be positioned over the inflamed area of the skin, either sticks to the sore area or is double layered and cause pressure, pain and discomfort for the users. Additionally, the sticky center makes it difficult to detach the patch from the sensitive area after use and will cause pain to the skin and the bunion instead of protecting the sensitive area. Some products add another layer to the center to make the center section not sticky. This double-layered center can also cause pressure and pain to the bunion instead of creating a comfortable environment.

In the following paragraphs, disclosed, among other things, is a bunion patch with a non-stick & a single-layer center, which provides a simple and practical treatment for podiatric care. In this disclosure, the words patch, pad, cushion, cover, and liner are interchangeable and each may be replaced by the others.

In the present disclosure, a bunion patch with non-stick & single-layer center is composed of a bottom layer and a top layer, each of which is made of a woven fabric or a non-woven sheet of any desired material or a combination of materials. The bottom layer has a hollow center and is designed to adhere to the user's skin and surround the user's sensitive or wounded area by placing such an upset area inside the patch's hollow center. The top layer is attached to the bottom layer such that to cover the hollow center and protect the sensitive area from above; however the portion of the surface of the top layer which is situated within the perimeter of the hollow center and which is located above the sensitive area of the skin, is free from any kind of adhesive and will not adhere to the user's skin even if it comes in contact with the upset area of skin.

The claimed bunion patch with non-stick & single-layer center will provide greater relief than conventional products because of the absence of stickiness in the center section and because of the single-layer center which does not strain the bunion or the sore area. The disclosed bunion patch may be provided in various shapes, sizes, colors and materials.

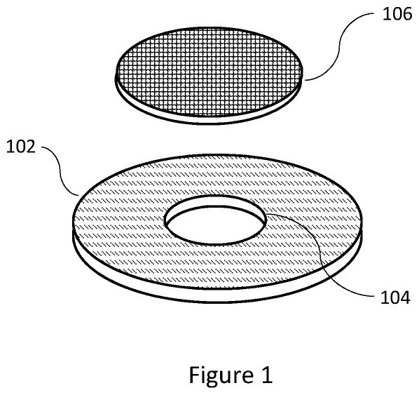

FIG. 1 shows an exploded view of an example structure of a bunion patch according to the disclosed apparatus and systems. In this figure the bottom layer 102 may be made of any desired woven or non-woven material(s). As seen in FIG. 1, there is an opening 104 inside the bottom layer 102. The lower surface of the bottom layer 102, or the surface away from the top layer 106, is configured to stick to the user's skin. With this configuration, the bottom layer 102 remains in place over the user's skin and surrounds the user's sensitive area, such as a bunion, and protects it from all sides.

In various embodiments, depending on the size and the shape of the sensitive area of the user, the size, the shape and the thickness of the bottom layer 102 and the size and the shape of the opening 104 may be different.

As shown in FIG. 1, the top layer 106, which is also fabricated from any desired woven or non-woven materials, covers the opening 104 of the bottom layer and further protects the user's sensitive area from above the sensitive area. In the claimed disclosure, only that portion of the surface of the top layer 106, which is attached to the bottom layer 102, uses some kind of adhesive. The rest of the surface area of the top layer 106, which remains within the perimeters of the opening 104, is free of adhesive. In various embodiments the size and/or the shape (excluding the opening) and/or the thickness of the top layer 106 may be the same as the bottom layer 102 and in other embodiments they can be different. Also, in different embodiments the materials of the top layer 106 may be the same as or different from the bottom layer 102.

In various embodiments the top layer 106 may be attached to the bottom layer 102 permanently or detachably for the user to be able to look under the top layer 106 and check her/his sensitive area or to apply needed medication before closing back the top layer 106. In some embodiments the surface area of the top layer within the perimeters of the opening/hole 104, may be coated with desired medication appropriate for the sensitive area. For example if the patch is applied to a toe corn, the medication used will be for the toe corn.

In some embodiments where the top layer 106 is detachably attached to the bottom layer 102, the top and the bottom layers may separately sold to the customer/user or be separately placed in the same package so that the user can apply new top layers 106 to the same bottom layer 102 every so often, especially if the user needs to renew the medication on the sensitive area every day or every several days. In such cases that the layers come separate from each other and must be attached together by the user, the adhesive may be covered by a non-stick paper and the user must remove the non-stick paper before attaching the layers 102 and 106 together.

In different embodiments the adhesive attaching the two layers may be initially on the top layer 106, on the bottom layer 102, on both layers 102 and 106, or on neither layer. In various embodiment the mechanism for attaching the two layers 102 and 106 may be Velcro® (or similar detachable technology) or the layers may be attached together by glue, resin, double-sided tape, heat, or by solvent chemicals. Yet in some embodiments the top layer 106 may be partially permanently attached and partially detachably attached to the bottom layer 102 so that a user can open the top layer 106 partially and apply medication through the partial opening to her sensitive area before closing it back.

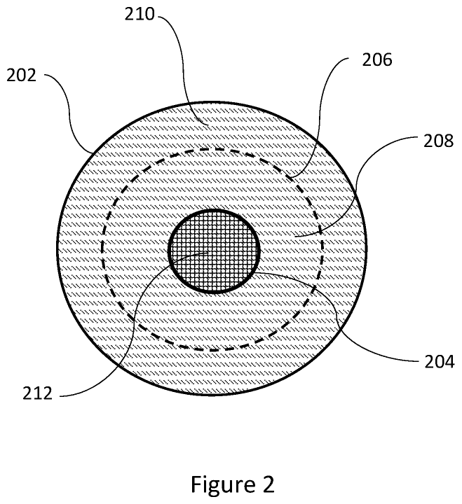

FIG. 2 illustrates an example of the assembly of the components of FIG. 1. This figure illustrates the assembly of a bunion or a toe-corn patch from below, where the patch touches and sticks to the user's skin. In FIG. 2, the bottom layer 202 consists of an opening/void 204 in its middle. The top layer 206 can be seen located behind the bottom layer 202 and is attached (permanently, detachably, or partially detachably) to the bottom layer 202, where the surfaces of both layers 202 and 206 overlap (“attach zone 208”). In some embodiments the attach zone 208 may be smaller than the total overlap surface, but in this figure they are the same. In other embodiments the surface area 212 of the top layer, which is positioned over the opening 204, has no adhesive but may bear some appropriate medication. In various embodiments the top layer 206 may have a different shape and/or size from the bottom layer 202 while in other embodiments the top layer 206 may have the same shape (excluding the opening) and size and thickness as the bottom layer 202.

As also mentioned in relation to FIG. 1, in various embodiments in the attach zone 208 the top layer 206 may be permanently attached to the bottom layer 202, while in other embodiments the layers 202 and 206 may be detachably attached and yet in some embodiments a part of the top layer 206 is permanently attached to the bottom layer 202 and the other part is detachably attached to the bottom part 202. The surface of the bottom layer 202, which is on the opposite side from the top layer 206 and which consists of areas 210 and 208, may be configured such that it completely or partially sticks to the user's skin and surrounds the user's bunion, toe corn, or any inflamed area while the surface area 212 of the top layer 206 covers the inflamed area from above.

Changes can be made to the claimed methods in light of the above Detailed Description. While the above description details certain embodiments of the disclosed methods and describes the best mode contemplated, no matter how detailed the above appears in text, the claimed methods can be practiced in many ways. Details of the system may vary considerably in its implementation details, while still being encompassed by the claimed methods disclosed herein.

Particular terminology used when describing certain features or aspects of the claimed methods should not be taken to imply that the terminology is being redefined herein to be restricted to any specific characteristics, features, or aspects of the claimed methods with which that terminology is associated. In general, the terms used in the following claims should not be construed to limit the claimed methods to the specific embodiments disclosed in the specification, unless the above Detailed Description section explicitly defines such terms. Accordingly, the actual scope of the claimed methods encompasses not only the disclosed embodiments, but also all equivalent ways of practicing or implementing the claimed methods.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B,” and also the phrase “A and/or B” will be understood to include the possibilities of “A” or “B” or “A and B.”

The above specification, examples, and data provide a complete description of the claimed methods. It is further understood that this disclosure is not limited to the disclosed embodiments, but is intended to cover various arrangements included within the spirit and scope of the broadest interpretation so as to encompass all such modifications and equivalent arrangements.

While the present disclosure has been described in connection with what is considered the most practical and preferred embodiment, it is understood that this disclosure is not limited to the disclosed embodiments, but is intended to cover various arrangements included within the spirit and scope of the broadest interpretation so as to encompass all such modifications and equivalent arrangements.

What is claimed is:

1. A cover for sensitive area of a user's skin, the cover comprising:
a top layer made of a single woven or a non-woven sheet of desired material(s);
a bottom layer made of a woven or a non-woven sheet of desired material(s), wherein the bottom layer forms an opening of a desired size and shape inside its periphery; and
wherein the top layer is permanently, detachably, or partially detachably attached to a first side of the bottom layer such that to cover the opening of the bottom layer, and wherein the bottom layer attaches to the user's skin, from a second side, such that to place the sensitive area of the user within the opening of the bottom layer while being covered by the top layer, and wherein a surface area of the top layer facing the sensitive area of the user has no adhesive.

2. The cover of claim 1, wherein the sensitive area of the user is a foot bunion, a toe corn, a foot callus, or a foot wart.

3. The cover of claim 1, wherein the top layer and/or the bottom layer is made of woven fabric, foam, non-woven fabric, gel, sponge or plastic film.

4. The cover of claim 1, wherein the bottom layer opening is a circular or an oval hole.

5. The cover of claim 1, wherein the top layer is partially permanently attached and partially detachably attached to the bottom layer.

6. The cover of claim 1, wherein the surface area of the top layer facing the sensitive area of the user is coated with medication.

7. A bunion patch comprising:
a top layer made of a single sheet of material(s);
a bottom layer made of a sheet of material(s), wherein the bottom layer forms an opening/void inside its perimeters; and
wherein the top layer is permanently or detachably attached to a first side of the bottom layer such that to cover the void inside the bottom layer, and wherein the bottom layer attaches to the user's skin, from a second side, such that to place the bunion of the user within the void of the bottom layer while being covered by the top layer, and wherein a surface area of the top layer facing the bunion of the user has no adhesive.

8. The bunion patch of claim 7, wherein the sheet of materials of the top and bottom layers are woven or non-woven.

9. The bunion patch of claim 7, wherein the top and/or the bottom layer is made of woven fabric, foam, non-woven fabric, gel, sponge or plastic film.

10. The bunion patch of claim 7, wherein the bottom layer opening/void is a circular or an oval hole.

11. The bunion patch of claim 7, wherein the top layer is partially permanently attached and partially detachably attached to the bottom layer.

12. The bunion patch of claim 7, wherein the surface area of the top layer facing the bunion of the user is coated with medication.

13. The bunion patch of claim 7, wherein air can pass through a portion of the top layer that covers the opening/void of the bottom layer.

14. A pad for sore area of a user's skin, the pad comprising:
a top layer made of a sheet of material(s);
a bottom layer made of a sheet of material(s), wherein the bottom layer creates a hole of a desired size and shape inside its periphery; and
wherein the top layer is permanently attached to a first side of the bottom layer such that to cover the hole of the bottom layer, and wherein the bottom layer attaches to the user's skin, from a second side, such that to place the sore area of the user within the hole of the bottom layer, and wherein a surface area of the top layer covering the hole of the bottom layer has no adhesive.

15. The pad of claim 14, wherein the sore area of the user is a foot bunion, a toe corn, a foot callus, or a foot wart.

16. The pad of claim 14, wherein the bottom layer is made of woven fabric, foam, non-woven fabric, gel, sponge or plastic film.

17. The pad of claim 14, wherein the top layer is made of woven fabric, foam, non-woven fabric, gel, sponge or plastic film.

18. The pad of claim 14, wherein the bottom layer hole is a circular or an oval hole.

19. The pad of claim 14, wherein air can pass through a portion of the top layer that covers the hole of the bottom layer.

20. The pad of claim 14, wherein a surface area of the top layer covering the hole of the bottom layer is coated with a desired medication.
